(19) United States
(12) Patent Application Publication
(10) Pub. No.: US 2012/0083800 A1
(43) Pub. Date: Apr. 5, 2012

(54) SYSTEMS AND METHODS FOR DEFINING A TRANSITION POINT OF A FOOT PEDAL OF AN OPHTHALMIC SURGERY SYSTEM

(76) Inventor: Lutz Andersohn, Glencoe, MO (US)

(21) Appl. No.: 12/897,084

(22) Filed: Oct. 4, 2010

(51) Int. Cl. A61B 19/00

(52) U.S. Cl. 606/130

(57) ABSTRACT

An ophthalmic surgery system includes an ophthalmic surgical console and a foot control assembly operably coupled to the ophthalmic surgical console and including a foot pedal depressible through a range of motion. The range of motion includes a first region for controlling a first function associated with a surgical handpiece operably coupled to the ophthalmic surgical console, a second region for controlling a second function associated with the surgical handpiece, and a transition point between the first region and the second region. The ophthalmic surgical console is configured to assign the transition point, in response to a setup routine, to an actual position of the foot pedal as defined by a user depressing the foot pedal.

### BACKGROUND

1. Field

The present disclosure is generally directed to defining at least one transition point of a foot pedal of an ophthalmic surgery system.

2. Description of the Related Art

The statements in this section merely provide background information related to the present disclosure and may not constitute prior art.

In eye surgery, use of ophthalmic surgical handpieces is commonly required to affect tissue within a patient's eye. Surgical handpieces may be able to provide a wide range of functions to the patient's eye, e.g., irrigation, aspiration, emulsification, etc. With the surgeon's hands occupied during a surgical procedure, a foot pedal is often utilized to control aspects of the one or more functions of one or more surgical handpieces. The foot pedal may include multiple regions, with each region providing control to an aspect of a function associated with a surgical handpiece. For example, a first region may control on/off of irrigation to a patient's eye, while a second region may linearly control a flow rate or aspiration, through the surgical handpiece, from the patient's eye.

In general, the multiple regions are bounded by transition points. Presently, various foot pedals include one or more fixed transition points, set during manufacturing of the foot pedals. In others, a transition point may be moveable by entering a specific angle of travel for each region. For example, through a user interface to a surgical system, a surgeon may numerically enter 0°-15° for the first region, 15°-25° for a second region, etc. A surgeon may have to go through numerous iterations of setting the transition points in order to select a comfortable or preferred setting.

Therefore, a need exists for an improved systems and methods for defining transition points between different regions of foot pedals for use in eye surgery.

### BRIEF DESCRIPTION OF THE DRAWINGS

The drawings described herein are for illustration purposes only and are not intended to limit the scope of the present disclosure in any way.

FIG. 1 is a plan view of a block diagram of an ophthalmic surgery system according to one embodiment of the present disclosure;

FIG. 2 is a diagram of a range of motion of a foot pedal included in the ophthalmic surgery system of FIG. 1; and

FIG. 3 is a diagram of a range of motion of a foot pedal according to one example embodiment of the present disclosure.

### DETAILED DESCRIPTION OF THE PREFERRED EMBODIMENT

The following description is merely exemplary in nature and is not intended to limit the present disclosure, application, or uses.

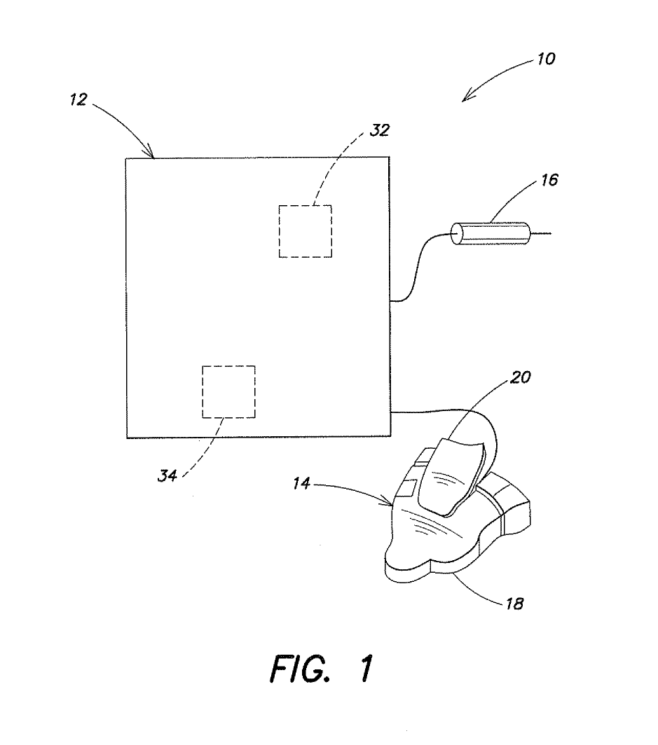

According to one embodiment of the present disclosure, an ophthalmic surgery system 10 is illustrated in FIG. 1. The ophthalmic surgery system 10 includes an ophthalmic surgical console 12 and a foot control assembly 14 operably coupled to the ophthalmic surgical console 12. The ophthalmic surgery system 10 further includes a surgical handpiece 16, usable for at least one ophthalmic surgical procedure. One or more surgical handpieces according to various embodiments of the present disclosure may be structured to perform various surgical functions, alone or in combination, suitable for posterior and/or anterior ophthalmic surgical procedures.

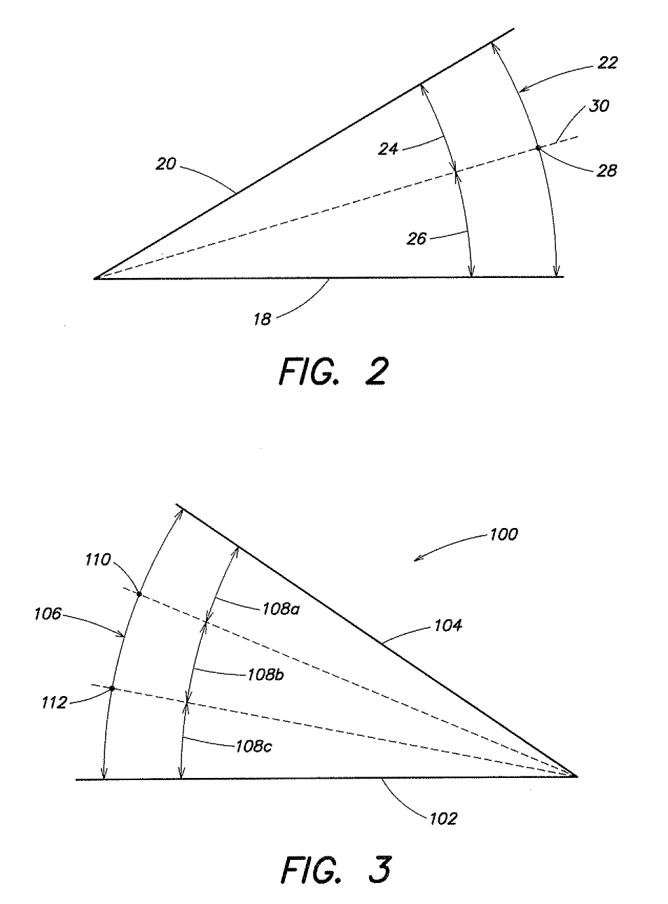

The foot control assembly 14 includes a base 18 and a foot pedal 20 pivotal relative to the base 18 through a range of motion 22. As shown in FIG. 2, the range of motion 22 includes a first region 24 for controlling a first function associated with the surgical handpiece 16 and a second region 26 for controlling a second function associated with the surgical handpiece 16. For example, a surgeon may increase/decrease depression of the foot pedal 20 in the first region to alter aspiration from a surgical site and increase/decrease depression of the foot pedal 20 in the second region to, linearly or non-linearly, alter an intensity of phacoemulsification or vitreous cutting to the surgical site. The range of motion 22 also includes a transition point 28 between the first region 24 and the second region 26.

The ophthalmic surgical console 12 is configured to assign the transition point 28, in response to a setup routine, to an actual position 30 of the foot pedal 20 within the range of motion 22 as defined by a user depressing the foot pedal 20. In this manner, the user, e.g., a surgeon, etc., is able to set one or more transition points based on “feel” of the foot pedal, thereby ensuring user comfort while operating the foot control assembly 14, eliminating user discomfort caused by one or more static transition points and/or minimizing time associated setting up transition points.

While the range of motion 22 of FIG. 2 includes the first and second regions 24, 26, it should be appreciated that in other embodiments, a range of motion may include a different numbers of regions. In at least one example embodiment, an ophthalmic surgical console is operably coupled to a surgical handpiece and a foot control assembly 100 having a base 102 and a foot pedal 104 movable through a range of motion 106. The range of motion 106 defines a first region 108a, a second region 108b, and a third region 108c, as shown in the diagram of FIG. 3. The regions 108a-c are separated by a first transition point 110 and a second transition point 112. Each of the regions 108a-c is associated with at least one of the functions of the surgical handpiece. For example, the first region 108a may be provided to control irrigation to a patient's eye. The second region 108b may be provided to simultaneously control irrigation and aspiration, and the third region 108c may be provided to simultaneously control aspiration and ultrasonic energy to the patient's eye. In other example embodiments, a range of motion may include four regions, five regions, or another suitable number of regions, etc. for control a number of different function associated with one or more surgical handpieces.

Further, although the region shown in FIGS. 2-3 are substantially equal, it should be appreciated that a region of a range of motion may be different from another region within the range of motion. In one example embodiment, a user may unevenly divide the range of motion into multiple region. A larger region may provide a user with a more range of control of the function associated with the region, while a smaller region may provide a less control of the function associated with the region. For example, controlling phacoemulsification over 15°, as compared to 5°, provides more control over an intensity of the phacoemulsification applied to a patient's eye.

Referring again to the ophthalmic surgical system 10, the setup routine is initiated by the ophthalmic surgical console 12 receiving a setup command from a surgeon or other user. The setup command is received via an input device 32 included in the ophthalmic surgical console 12, e.g., a touchscreen display, a button, a switch, a keyboard, etc. In various other embodiments, a setup routine may be automatically initiated by an ophthalmic surgical console, based on one or more conditions. For example, a setup routine may be automatically initiated by coupling a foot control assembly, at least one surgical handpiece, one or more aspiration/irrigation lines, etc. to an ophthalmic surgical console. In other examples, a setup routine may be initiated, automatically, by selecting a surgical procedure or at power-up of an ophthalmic surgical console, a surgical handpiece, and/or a foot control assembly, etc.

Once the setup routine is initiated, the foot pedal 20 is depressed by the user to define the actual position 30 (dotted line). When the foot pedal 20 is in the actual position 30, the ophthalmic surgical console receives a mark command from the user, via an input device. In turn, the ophthalmic surgical console 12 assigns the transition point 28, between the first region 24 and the second region 26, to the actual position 30 of the foot pedal 20. It should be appreciated that a transition point may be assigned to an actual position of a foot pedal in a different manner in other embodiments. In one example embodiment, after initiation of a setup routine, an ophthalmic surgical console may monitor depression of a foot pedal by a user to identify a lowest point of depression. When the foot pedal is released, the ophthalmic surgical console may understand the lowest point of depression to be an actual position of the foot pedal, and assign a transition position between different regions to the actual position.

For a range of motion having more than two regions, it should be appreciated that additional inputs may be received by one or more input devices of an ophthalmic surgical console to indicate which multiple transition points is being set by the user and the actual position associated with said transition point. In other embodiments of the present disclosure, an ophthalmic surgical console may receive an input to allocate a region to a particular function of a surgical handpiece operably coupled to the ophthalmic surgical console. Accordingly, a range of motion of a foot pedal may be substantially customized to particular preferences and/or comforts of a user. It should further be appreciated that a user may initiate a setup routine and exit from a setup routine, through an input to an ophthalmic surgical console, such that the user may initiate, re-initiate, and/or exit from the setup routine, throughout a surgical procedure.

According to various embodiments of the present disclosure, a foot pedal may be moveable in one or more planes. As shown in FIG. 2, the movement of the foot pedal 20 includes a pitch movement. Additionally, the movement of the foot pedal 20 includes a yaw movement, not shown. In other example embodiments, a movement of a foot pedal may include only a yaw movement or only pitch movement. The pitch and the yaw may be employed in various regions to control a variety of functions associated with a surgical handpiece. A pitch movement may define a vertical range of motion having multiple regions, and a yaw movement may define a horizontal range of motion having multiple regions. In this manner, numerous different regions may be defined to control numerous different functions associated with a surgical handpiece.

As shown in FIG. 2, the foot pedal 20 is pivotal relative to the base 18 of the foot control assembly 14. In this particular embodiment, the range of motion includes about 40° of pivot. It should be appreciated that, in other embodiments, a foot pedal may employ different ranges of motion, e.g., a pitch range of motion from about 90° to about 10°, yaw range of motion from about 180° to about 10°, or various combinations of pitch and/or yaw ranges of motions, etc. In one example embodiment, foot pedal range of motion may include a pitch range of motion may of about 50° and a yaw range of motion of about 20°.

In one embodiment of the present disclosure, when setting a transition position in a pitch range of motion, a user may depress a foot pedal to an actual position, and then provide a mark command to an ophthalmic surgical console by moving the foot pedal in a yaw range of motion. In this manner, the user is able to provide a hands-free mark command, during a setup routine. Alternatively or additionally, a transition point in a yaw range of motion may be marked by a movement in a pitch range of motion. In other embodiments the transition positions may input a user mark command with any acceptable input device including voice command, touch screen, or dedicated button on the console of the foot pedal itself. Other input devices and methods not specifically disclosed may also be used to input a mark command during set up of the foot control assembly.

Further, within a range of motion, movement of the foot pedal may be subject to variable resistance to provide feedback to a user. In one example, resistance to depression of a foot pedal may increase as depression increases. Further still, an electronic clutch, brake or other programmable resistance devices known in the art may be employed in a conventional manner in a foot control assembly, and responsive to an ophthalmic surgical console, to provide resistance indication of a transition point, i.e., a detent. Other feedback mechanism, e.g., audible, visual, vibrational, etc., may be incorporated in a foot control assembly to signal one or more transition points to a user, during use of the foot control assembly.

A foot control assembly including other buttons, switches, and/or keys, etc. to provide further control to a surgeon. Referring to FIG. 1, the foot control assembly 14 includes multiple foot switches, which may be programmed to toggle various modes and/or functions of the ophthalmic surgery system 10. In one example, a button may be disposed on a foot control assembly adjacent to a foot pedal to alter a surgical mode of the system between fragmentation, vitrectomy, scissor, extrusion, etc.

The ophthalmic surgical console 12 includes memory 34 suitable for storing the setup routine, such that the ophthalmic surgical console 12 may execute the setup routine. It should be appreciated that a number of different types of memory may be included, fixedly or removable, in the ophthalmic surgical console 12. In other embodiments, an ophthalmic surgical console may include a memory suitable for storing one or more transition points of one or more users for one of more surgical procedures. In this manner, a user may be able to recall one or more transition points for a surgical procedure, e.g., phacoemulsification, etc., without initiating an additional setup routine. The memory may be suitable to store one or more transition points of a foot pedal for one or multiple days, or longer depending on at least a user preferences.

According to one example embodiment of the presented disclosure, a method for defining a first region and a second region of foot pedal of a foot control assembly operably coupled to an ophthalmic surgical console is disclosed. The foot pedal is movable in a range of motion including a transition point between the first region and the second region. The method includes receiving an input to initiate a setup routine, measuring a desired position of the foot pedal within the range of motion, and assigning said position of the foot pedal as the transition point between the first region and second region.

Although several aspects of the present disclosure have been described above with reference to foot control assemblies, it should be understood that various aspects of the present disclosure are not limited to foot control assemblies, and can be applied to a variety of other ophthalmic surgery systems and various components included therein.

By implementing any or all of the teachings described above, a number of benefits and advantages can be attained including improved reliability, reduced down time, elimination or reduction of redundant components or systems, avoiding unnecessary or premature replacement of components or systems, and a reduction in overall system and operating costs.

We claim:

1. An ophthalmic surgery system comprising:
an ophthalmic surgical console;
a foot control assembly operably coupled to the ophthalmic surgical console and including a foot pedal depressible through a range of motion;
the range of motion including a first region for controlling a first function associated with a surgical handpiece operably coupled to the ophthalmic surgical console, a second region for controlling a second function associated with the surgical handpiece, and a transition point between the first region and the second region; and
wherein the ophthalmic surgical console is configured to assign the transition point, in response to a setup routine, to an actual position of the foot pedal as defined by a user depressing the foot pedal.

2. The ophthalmic surgery system of claim 1, wherein the setup routine includes a mark command received by the ophthalmic surgical console to indicate the foot pedal is disposed at said actual position.

3. The ophthalmic surgery system of claim 1, wherein the ophthalmic surgical console includes an input device to receive a setup command from a user, the setup command initiating the setup routine.

4. The ophthalmic surgery system of claim 1, wherein the ophthalmic surgical console includes a memory, said setup routine being stored in memory and executable by the ophthalmic surgical console.

5. The ophthalmic surgery system of claim 1, wherein the range of motion includes a third region and a second transition point between the second region and the third region, the ophthalmic surgical console being configured to assign the second transition point, in response to said setup command, to a second actual position of the foot pedal within the range of motion as defined by a user depressing the foot pedal.

6. The ophthalmic surgery system of claim 1, wherein the range of motion includes at least one of a pitch range of motion and a yaw range of motion.

7. The ophthalmic surgery system of claim 1, wherein the ophthalmic surgical console is configured to automatically initiate the setup routine in response to a surgical handpiece and/or the foot control assembly being coupled to the ophthalmic surgical console.

8. A method of defining a transition point between a first region and a second region of foot pedal of a foot control assembly operably coupled to an ophthalmic surgical console, the foot pedal movable in a range of motion including the first region and the second region, the method comprising:
initiating a setup routine; and
assigning the transition point between the first region and the second region to an actual position of the foot pedal as defined by a user depressing the foot pedal.

9. The method of claim 8, further comprising receiving a mark command to an input device of the ophthalmic surgical console, indicating the foot pedal is disposed in said actual position.

10. The method of claim 8, wherein the range of motion includes a pitch range of motion and a yaw range of motion.

11. The method of claim 8, furthering comprising receiving a signal indicative of the actual position of the foot pedal within the range of motion.

12. The method of claim 8, wherein initiating the setup routine includes receiving a setup command from a user.

13. The method of claim 8, further comprising assigning a second transition point between the second region and a third region to a second actual position of the foot pedal as defined by a user depressing the foot pedal.

14. An ophthalmic surgery system comprising an ophthalmic surgical console having a memory, a foot control assembly operatively coupled to the ophthalmic surgical console, the foot control assembly having a foot pedal movable through a range of motion, and a program stored in the memory and executable by the ophthalmic surgical console for initiating a setup routine and assigning a transition point between a first region and a second region, in response to a setup routine, to an actual position of the foot pedal as defined by a user depressing the foot pedal.
